(19) United States
(12) Patent Application Publication
(10) Pub. No.: US 2016/0213950 A1
(43) Pub. Date: Jul. 28, 2016

(54) PARTICLE BEAM THERAPY SYSTEM, AND METHOD FOR OPERATING PARTICLE BEAM THERAPY SYSTEM

(71) Applicant: Hitachi, Ltd., Tokyo (JP)

(72) Inventors: Futaro EBINA, Tokyo (JP); Hideaki NISHIUCHI, Tokyo (JP)

(21) Appl. No.: 14/976,089

(22) Filed: Dec. 21, 2015

(30) Foreign Application Priority Data
Jan. 28, 2015 (JP) 2015-013869

(51) Int. Cl. A61N 5/10

(52) U.S. Cl. CPC A61N 5/1048; A61N 5/1067

(57) ABSTRACT

When a beam acceleration high frequency control section turns on an accelerating cavity in synchronization with a beam irradiation on state, the beam acceleration high frequency control section rapidly increases an amplitude value of an applied voltage of the accelerating cavity. The control section rapidly decreases the amplitude value of the applied voltage of the accelerating cavity before beam irradiation off timing. When the control section turns off the accelerating cavity, the control section gradually decreases the amplitude value of the applied voltage of the accelerating cavity before the irradiation off timing. When the control section turns on the accelerating cavity and starts the beam irradiation, the control section gradually increases the applied voltage of the accelerating cavity.

### BACKGROUND OF THE INVENTION

1. Field of the Invention

The present invention relates to a particle beam radiotherapy system and a control method for the same.

2. Description of the Related Art

In JP-2009-45170-A, a particle beam radiotherapy system that derives an irradiation beam suitable for a particle beam radiotherapy implemented by the spot scanning technique and that is inexpensively accomplished is disclosed. The particle beam radiotherapy system includes a synchrotron that accelerates a charged particle beam at a high frequency acceleration voltage applied to an accelerating cavity until the charged particle beam has predetermined energy and causes an emission unit to emit the charged particle beam that exceeds its stable limit in a high frequency electromagnetic field applied to the emission unit, a beam transport that guides the charged particle beam emitted from the synchrotron to a radiotherapy chamber, and an irradiation unit that irradiates a patient with the charged particle beam corresponding to a shape of a diseased part of a patient in the radiotherapy chamber. The particle beam radiotherapy system also includes a control unit that turns on the high frequency electromagnetic field applied to the emission unit when the charged particle beam is supplied to the irradiation unit, that turns off the high frequency electromagnetic field applied to the emission unit and causes an electromagnet disposed in the beam transport or the synchrotron to stop supplying the charged particle beam when the charged particle beam is stopped to be supplied to the irradiation unit, and that turns off the high frequency acceleration voltage applied to the accelerating cavity in synchronization with on/off states of the high frequency electromagnetic field applied to the emission unit.

### SUMMARY OF THE INVENTION

In the synchrotron that composes the particle beam radiotherapy system described in JP-2009-45170-A, while a beam extraction unit such as a high frequency kicker is turned on (beam irradiation on timing), the accelerating cavity is turned on. While the beam extraction unit is turned off (during beam irradiation off timing), the accelerating cavity is turned off. As a result, after the beam stop timing, oscillations of particles of the circling beam (synchrotron oscillations) stop. Since the amount of circling beam that is extracted due to the synchrotron oscillations decreases, the irradiation dose can be accurately controlled.

In the particle beam radiotherapy system described in JP-2009-45170-A, however, after the accelerating cavity is turned off until particles of a circling beam uniformly distributes in their longitudinal direction, the momentum of particles of the circling beam changes due to the space charge effect. As a result, after the beam irradiation off timing, a beam is slightly extracted from the synchrotron. Thus, the beam irradiation needs to be more accurately performed.

Therefore, an object of the present invention is to provide a particle beam radiotherapy system and a control method for the same that allow the dose of a beam to be controlled more accurately than the system and method in the past.

To solve the foregoing problem, structures described for example in the claims are used.

The present invention includes a plurality of units to solve the foregoing problem, and examples thereof include a particle beam radiotherapy system, including: a synchrotron that accelerates and emits a charged particle beam; an irradiation unit that irradiates an irradiation target with the charged particle beam emitted from the synchrotron; a beam transport that transports the charged particle beam from the synchrotron to the irradiation unit; and a control unit that controls the synchrotron, the beam transport, and the irradiation unit, wherein the synchrotron includes an accelerating cavity that accelerates the charged particle beam at a high frequency acceleration voltage until the charged particle beam has predetermined energy and an extraction unit that extracts the charged particle beam from the synchrotron, and wherein the control unit includes a beam acceleration high frequency control section that decreases an amplitude value of a high frequency voltage applied to the accelerating cavity while the extraction unit performs extraction control for the charged particle beam.

According to the present invention, the dose of a beam can be controlled more accurately than the system and method in the past.

Other problems, structures, and effects of the present invention will become apparent from the description of preferred embodiments that follow.

### BRIEF DESCRIPTION OF THE DRAWINGS

FIG. 1 is a diagram showing an example of a structure of a particle beam radiotherapy system according to a first embodiment of the present invention;

FIG. 2 is a schematic diagram showing an irradiation field forming unit of the particle beam radiotherapy system according to the first embodiment of the present invention;

FIG. 3 is a time chart showing a beam irradiation control method in the past implemented by the scanning irradiation method;

FIG. 4 is a time chart showing the beam irradiation control method according to the first embodiment of the present invention; and

FIG. 5 is a time chart showing a beam irradiation control method according to a second embodiment of the present invention.

### DESCRIPTION OF THE PREFERRED EMBODIMENTS

Next, with reference to the accompanying drawings, a particle beam radiotherapy system and a control system for the same according to embodiments of the present invention will be described.

### First Embodiment

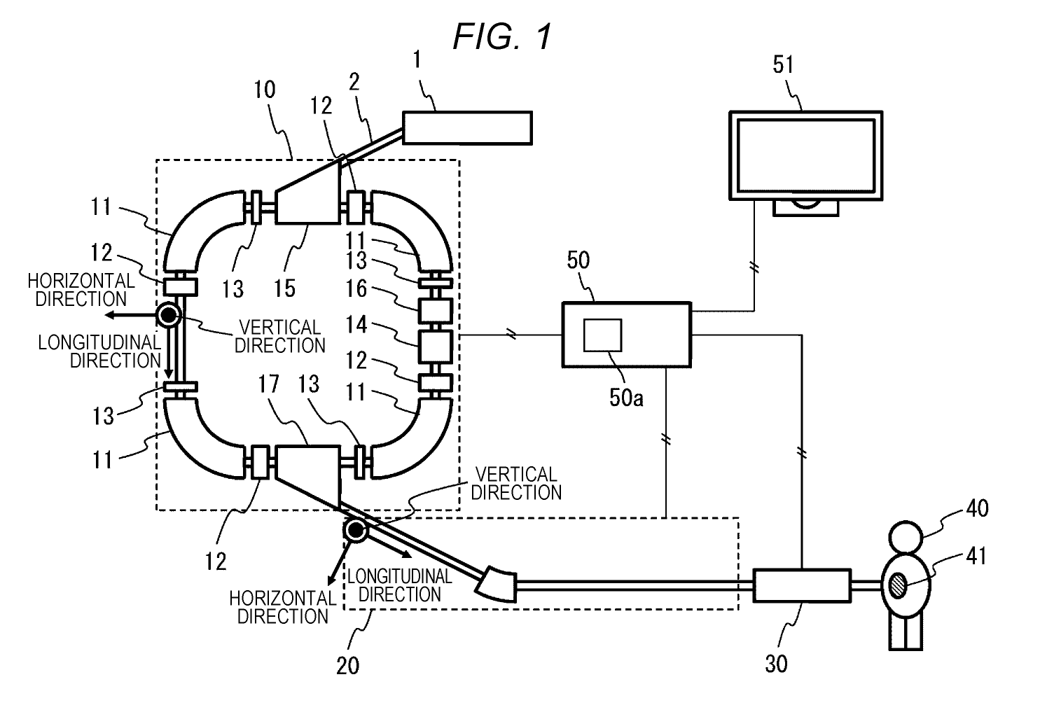

With reference to FIGS. 1 to 4, a particle beam radiotherapy system and a control method for the same according to a first embodiment of the present invention will be described. The present embodiment is for example a particle beam radiotherapy system that can accurately control the dose of a beam. FIG. 1 is a diagram showing an example of a particle beam radiotherapy system according to the present embodiment.

In the particle beam radiotherapy system according to the present embodiment shown in FIG. 1, a charged particle beam (hereinafter referred to as the beam) is injected from an injection unit 1 into a synchrotron 10. The synchrotron 10 accelerates the beam until it has predetermined kinetic energy (hereinafter, kinetic energy is simply referred to as energy). Thereafter, the beam is extracted from the synchrotron 10. A diseased part 41 of a patient 40 is irradiated with the beam. The particle beam radiotherapy system includes the synchrotron 10, an irradiation field forming unit 30, a high energy beam transport 20, and a control unit 50.

The injection unit 1 is for example a linear accelerator (linac) that accelerates the beam generated by an ion source (not shown) until it has energy (hereinafter referred to as injection energy) with which the beam is injected into the synchrotron 10.

The charged particle beam extracted from the injection unit 1 is injected into the synchrotron 10 through a low energy beam transport 2 and an inflector for injection 15.

The synchrotron 10 includes the inflector for injection 15, a bending magnet 11, a quadrupole magnet 12, an extupole magnet 13, an accelerating cavity 14, a high frequency voltage applying and extracting unit 16, and a deflector for extraction 17.

The bending magnet 11 deflects a beam that circles in the synchrotron 10 (hereinafter this beam is referred to as the circling beam) so as to form a predetermined circling orbit (hereinafter referred to as the circling beam orbit).

In FIG. 1, a direction along a longitudinal direction of the circling beam is referred to as the longitudinal direction (the direction in which the beam travels is plus), a direction perpendicular to the longitudinal direction and along a radial direction of the bending magnet 11 is referred to as the horizontal direction (the outer direction of the synchrotron is plus), and a direction perpendicular to both the longitudinal direction and the horizontal direction is referred to as the vertical direction (the inward direction of the drawing is plus). A designed circling beam orbit of the synchrotron 10 is referred to as the center orbit. Particles of a circling beam oscillate around the center orbit in the horizontal and vertical directions. These oscillations are referred to as the betatron oscillations. The frequency of the betatron oscillations per one turn of the synchrotron is referred to as the tune.

The quadrupole magnet 12 applies a converging force or a diverging force to the circling beam so that the tune of the circling beam is kept at a value with which the circling beam becomes stable.

The accelerating cavity 14 applies a high frequency voltage (hereinafter referred to as the acceleration voltage) to the circling beam in the longitudinal direction, captures the circling beam at a predetermined phase in the longitudinal direction (hereinafter referred to as high frequency capture), and accelerates the circling beam until it has predetermined energy.

The momentum of the high-frequency-captured particles of the circling beam oscillates around the designed momentum (hereinafter referred to as the center momentum). These oscillations are referred to as the synchrotron oscillations.

While the synchrotron 10 accelerates the circling beam, the synchrotron 10 increases the amount of magnetization of the bending magnet 11 and that of the quadrupole magnet 12 in proportion to the momentum of the circling beam, controls the frequency of the acceleration voltage (hereinafter referred to as the acceleration frequency) to an appropriate value, and keeps the orbit of the circling beam and the tune of the circling beam constant.

After the synchrotron 10 has accelerated the circling beam, the synchrotron 10 changes the amount of magnetization of the quadrupole magnet 12 so that the horizontal tune of the circling beam becomes a value with which the circling beam becomes unstable (hereinafter this value is referred to as the resonance line). In addition, the synchrotron 10 magnetizes the sextupole magnet 13 so as to apply a magnetic field having an intensity proportional to the square of the distance from the center orbit (hereinafter this magnetic field is referred to as the sextupole magnetic field) to the circling beam so as to form a stable limit of the horizontal betatron oscillations on a phase space defined by the horizontal positions and the orientations of particles of the circling beam (hereinafter the stable limit is referred to as the separatrix).

The high frequency voltage applying and extracting unit 16 applies a high frequency voltage in the horizontal direction that synchronizes with the horizontal tune to the circling beam so as to increase the amplitude of the horizontal betatron oscillations of particles of the circling beam. As a result, the amplitude of the horizontal betatron oscillation increases. When particles of the circling beam exceed the separatrix, the amplitude of the horizontal betatron oscillations rapidly increases and is injected into the deflector for extraction 17.

The deflector for extraction 17 deflects particles of the injected circling beam in the horizontal direction and extracts them out of the synchrotron 10.

The beam extracted from the synchrotron 10 (hereinafter this beam is referred to as the extracted beam) is supplied to the high energy beam transport 20 and the irradiation field forming unit 30. Thereafter, the diseased part 41 is irradiated with the extracted beam. The coordinate systems of the high energy beam transport 20 and the irradiation field forming unit 30 are based on that of the synchrotron 10. The position of the diseased part 41 in the beam longitudinal direction is referred to as the irradiation point.

The high energy beam transport 20 connects the synchrotron 10 and the irradiation field forming unit 30. The high energy beam transport 20 conveys the extracted beam emitted from the synchrotron 10 to the irradiation field forming unit 30. The high energy beam transport 20 includes a deflection magnet that deflects the extracted beam toward the patient 40, a quadrupole magnet that applies a converging force or a diverging force to the extracted beam, and a steering magnet that adjusts the orbit of the extracted beam.

The irradiation field forming unit 30 trims the beam conveyed from the high energy beam transport 20 and forms a distribution of the irradiation dose based on the shape of the diseased part 41 (hereinafter this distribution is referred to as the irradiation field). The particle beam radiotherapy system according to the present embodiment implements the scanning irradiation method that causes a scanning electromagnet to scan the diseased part 41 so as to form the irradiation field.

After the circling beam has been extracted, the synchrotron 10 changes the amount of magnetization of the bending magnet 11, the amount of magnetization of the quadrupole magnet 12, and the acceleration frequency to those for which the beam is injected into the synchrotron 10 so that the next beam is injected into the synchrotron 10. The time after the beam is injected into the synchrotron 10 until the next beam is injected into the synchrotron 10 is referred to as the period of the synchrotron 10.

The control unit 50 is connected to the irradiation field forming unit 30, the high energy beam transport 20, and the synchrotron 10 so that the control unit 50 controls components that compose the irradiation field forming unit 30, the high energy beam transport 20, and the synchrotron 10. The control unit 50 includes a beam acceleration high frequency control section 50a. When the accelerating cavity 14 is turned on in synchronization with the beam irradiation on state, the beam acceleration high frequency control section 50a rapidly increases the amplitude value of the applied voltage of the accelerating cavity 14. In addition, while the high frequency voltage applying unit 16 performs the beam extraction control for the charged particle beam (during the beam irradiation on state), the beam acceleration high frequency control section 50a rapidly decreases the amplitude value of the applied high frequency voltage of the accelerating cavity 14.

When the amplitude value of the applied voltage of the accelerating cavity 14 is rapidly increased, the amplitude value of the applied voltage of the accelerating cavity 14 is increased, substantially the voltage is turned on, for the time particles of the circling beam circle one time in the synchrotron 10. Likewise, when the amplitude value of the applied voltage of the accelerating cavity 14 is rapidly decreased, the amplitude value of the applied voltage of the accelerating cavity 14 is decreased, substantially the voltage is turned off, for the time particles of the circling beam circles one time in the synchrotron 10.

A display unit 51 is connected to the control unit 50. While the synchrotron 10 is operated, time changes of a beam irradiation enable signal and time changes of the applied voltage of the accelerating cavity 14 are displayed on the same screen. With reference to these signals displayed on the display unit 51, a technician or a user of the particle beam radiotherapy system can check that the accelerating cavity 14 has been turned off before the irradiation off timing. Alternatively, the time changes of the irradiation enable signal and the time changes of the applied voltage of the accelerating cavity 14 may be stored by the control unit 50 as a file in a recording unit (not shown) such as a hard disk drive of the control unit 50. With reference to the file, the technician or the user of the particle beam radiotherapy system may check that the accelerating cavity 14 has been turned off before the irradiation off timing.

The particle beam radiotherapy system according to the present embodiment repeats acceleration, extraction, and irradiation of the beam until beam irradiation designated by a radiotherapy planning unit (not shown) is completed.

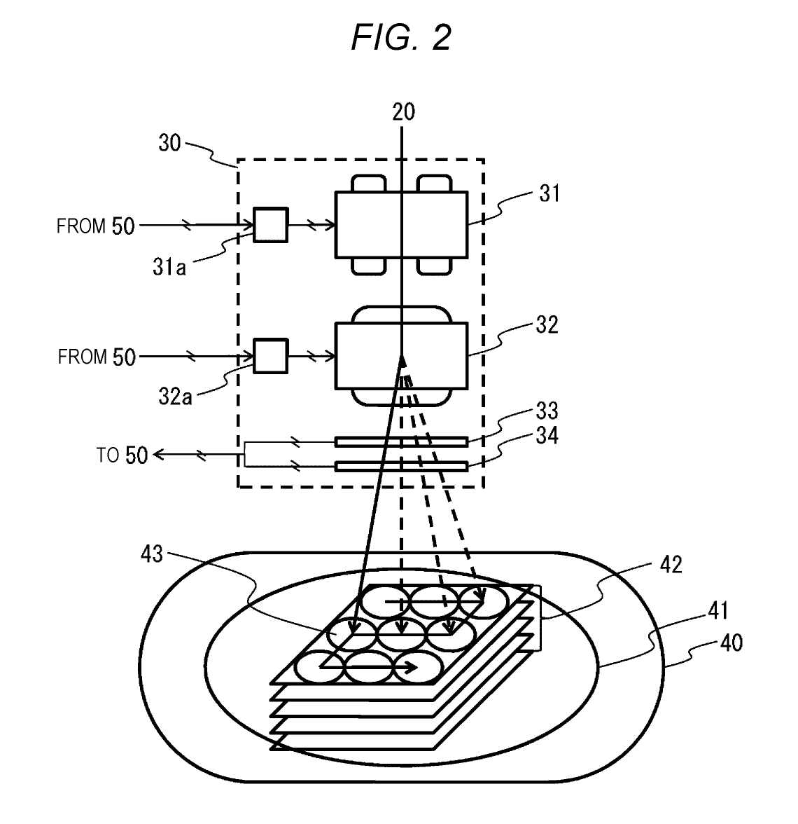

Next, with reference to FIG. 2, a method for forming an irradiation field corresponding to the shape of the diseased part 41 will be described. The irradiation field is formed by the irradiation field forming unit 30. FIG. 2 is a schematic diagram showing a structure of the irradiation field forming unit 30.

In this example, the irradiation field forming unit 30 includes scanning electromagnets 31 and 32, a dose monitor 33, and an irradiation beam position monitor 34. The scanning electromagnets 31 and 32 are connected to scanning electromagnet power supplies 31a and 32a, respectively. The scanning electromagnet power supplies 31a and 32a, the dose monitor 33, and the irradiation beam position monitor 34 are connected to the control unit 50.

The irradiation field is divided into a plurality of irradiation layers 42 in the beam longitudinal direction (depth direction). In addition, each of the irradiation layers 42 is divided into a plurality of irradiation spots 43 that distribute on a plane perpendicular to the beam longitudinal direction. It depends on energy of the irradiation beam that with which beam an irradiation layer 42 is irradiated, namely that the position (range) in the beam longitudinal direction having the maximum energy in the body of the patient 40 is at which irradiation layer 42. The synchrotron 10 adjusts energy of the circling beam that has been accelerated, namely energy of irradiation beam, and decides the irradiation layer 42 irradiated with the beam. Information of the irradiation layer 42 and the irradiation spot 43 that compose the irradiation field is generated by the radiotherapy planning unit (not shown) as designated by the user of the particle beam radiotherapy system.

The control unit 50 reads the information of the irradiation layer 42 and the irradiation spot 43, which compose the irradiation field, from the radiotherapy planning unit and controls the synchrotron 10 to accelerate the circling beam until it has energy corresponding to the irradiation layer 42 initially irradiated with the beam. After the circling beam has been accelerated and has been ready to be extracted from the synchrotron 10, the synchrotron 10 outputs an irradiation standby signal to the control unit 50.

When the control unit 50 receives the irradiation standby signal, the control unit 50 magnetizes the scanning electromagnets 31 and 32 with a predetermined amount of magnetization that causes the beam to be deflected toward the first irradiation spot 43. After the scanning electromagnets 31 and 32 have been magnetized, the control unit 50 turns on the irradiation enable signal.

When the irradiation enable signal is turned on, the high frequency voltage applying and extracting unit 16 of the synchrotron 10 is turned on. As a result, the high frequency voltage in the horizontal direction is applied to the circling beam and extracted out of the synchrotron 10. When the high frequency voltage applying and extracting unit 16 is turned on, it applies the high frequency voltage to the circling beam. In other words, the high frequency voltage applying and extracting unit 16 performs the beam extraction control. Likewise, when the high frequency voltage applying and extracting unit 16 is turned off, it does not apply the high frequency voltage to the circling beam. In other words, the high frequency voltage applying and extracting unit 16 does not perform the beam extraction control.

The beam extracted from the synchrotron 10 is directed to the high energy beam transport 20. Thereafter, the beam is deflected in magnetic fields generated by the scanning electromagnets 31 and 32 so that the first irradiation spot 43 is irradiated with the beam.

While the irradiation is performed, the irradiation beam position monitor 34 measures the irradiation position of the beam deflected by the scanning electromagnets 31 and 32 and determines whether or not the measured result of the position of the irradiation beam matches the position of the irradiation spot 43 designated by a radiotherapy plan. If the real irradiation position does not match the planned position, the control unit 50 turns off the irradiation enable signal so as to stop extracting the beam from the synchrotron 10.

In addition, while the irradiation is performed, the dose monitor 33 measures the irradiation dose with which each of the irradiation spots 43 is irradiated (hereinafter, the dose of a beam is referred to as the irradiation dose). When the irradiation dose for the current irradiation spot reaches a target value designated by the radiotherapy plan, the control unit 50 turns off the irradiation enable signal so as to stop extracting the beam from the synchrotron 10.

After the irradiation dose has reached the target value and the control unit 50 has stopped extracting the beam, the control unit 50 controls the amount of magnetization of the scanning electromagnets 31 and 32 so as to deflect the beam to the next irradiation spot 43 and resume beam extraction from the synchrotron 10. The control unit 50 successively performs the beam irradiation for all irradiation spots 43 of the current irradiation layer 42. Thereafter, like the first irradiation layer 42, the control unit 50 successively performs the beam irradiation for all irradiation layers 42 that compose the irradiation field and forms the irradiation field corresponding to the shape of the diseased part 41 of the patient 40.

Next, a control method for improving accuracy of the irradiation dose according to the present embodiment will be described. In this case, when the accuracy of the irradiation dose is high, the difference between the irradiation dose for each irradiation spot and the target dose is small.

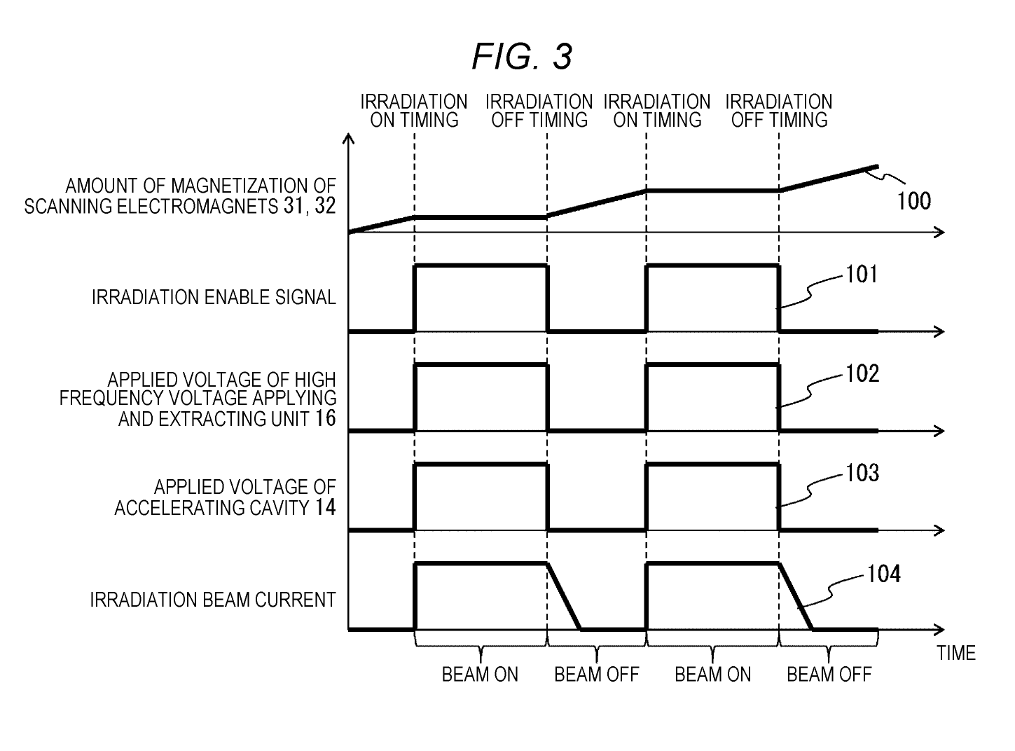

First, with reference to FIG. 3, a beam irradiation on/off control method implemented by the scanning irradiation method in the past will be described. FIG. 3 is a time chart showing the beam irradiation control method in the past implemented by the scanning irradiation method.

In FIG. 3, the horizontal axis represents time and the vertical axis successively represents time changes of the amount of magnetization of the scanning electromagnets 31 and 32 (polygonal line 100), the beam irradiation enable signal of the control unit 50 (polygonal line 101), the amplitude of the high frequency voltage in the horizontal direction and applied to the circling beam by the high frequency voltage applying and extracting unit 16 (hereinafter, this amplitude is referred to as the applied voltage of the high frequency voltage applying and extracting unit 16) (polygonal line 102), the amplitude of the high frequency voltage in the longitudinal direction and applied to the circling beam by the accelerating cavity 14 (hereinafter this amplitude is referred to as the applied voltage of the accelerating cavity 14) (polygonal line 103), and the current of the beam extracted from the synchrotron 10 and with which the diseased part 41 is irradiated (hereinafter this current is referred to as the irradiation beam current) (polygonal line 104). Although the irradiation nozzle according to the present embodiment is provided with two scanning electromagnets 31 and 32, FIG. 3 schematically shows the amount of magnetization of the two scanning electromagnets 31 and 32 with one polygonal line 100.

In FIG. 3, after the scanning electromagnets 31 and 32 have been magnetized with predetermined amounts of magnetization and the irradiation enable signal 101 has been turned on, the high frequency voltage applying and extracting unit 16 is turned on. As a result, the high frequency voltage in the horizontal direction is applied to the circling beam and the diseased part 41 is irradiated with the beam. While the beam irradiation is performed, the acceleration voltage of the accelerating cavity 14 is applied to the circling beam.

When the irradiation dose reaches the target dose for the current irradiation spot, the irradiation enable signal 101 is turned off and the high frequency voltage applying and extracting unit 16 is turned off. As a result, the beam irradiation for the diseased part 41 is stopped.

While the acceleration voltage is applied to the circling beam, the momentum of each of particles of the charged beam that compose the circling beam changes due to the synchrotron oscillations. When the momentum of particles of the circling beam changes, the horizontal tune of particles of the circling beam changes due to the momentum of the synchrotron 10 and the chromaticity of the horizontal tune (this chromaticity is referred to as the horizontal chromaticity). At this point, when the tune of particles of the circling beam changes and approaches the resonance line used for beam extraction, the separatrix of the particles of the circling beam decreases. As a result, particles of the circling beam may move outside the separatrix. Thus, particles of the circling beam may be extracted. Thus, even if the high frequency voltage applying and extracting unit 16 is turned off, while the accelerating cavity 14 is turned on, the circling beam is likely to be extracted. In this case, a difference between the irradiation dose and the target dose prevents the irradiation dose from being accurately controlled.

Thus, in the scanning irradiation method in the past, when the high frequency voltage applying and extracting unit 16 is turned off, the accelerating cavity 14 is turned off. In other words, the high frequency voltage of the accelerating cavity 14 is stopped so as to stop the synchrotron oscillations of particles of the circling beam and prevent the circling beam from being extracted after the beam irradiation stop timing. However, while the accelerating cavity 14 is turned off, although the synchrotron oscillations of particles of the circling beam are stopped, when the accelerating cavity 14 is turned off for example, 100 ms, that is much longer than the time for example, 5 ms, necessary to change the amount of magnetization of the scanning electromagnets, interactions of particles of the circling beam cause them to become unstable. As a result, the circling beam is likely to be lost.

Thus, according to the scanning irradiation method in the past, when the irradiation enable signal is turned on next time (at the irradiation on timing), the accelerating cavity 14 is turned on. As a result, the synchrotron oscillations of particles of the circling beam are resumed so as to prevent the circling beam from becoming unstable. Thus, while the irradiation enable signal is turned on, even if the circling beam is extracted due to a decrease of the separatrix, the accuracy of the irradiation dose is not lowered.

Thus, according to the scanning irradiation method in the past, the accelerating cavity 14 is turned on and off in synchronization with the on/off states of the irradiation enable signal (of the high frequency voltage applying unit 16). As a result, while the irradiation enable signal is turned off, the amount of the beam extracted from the synchrotron 10 is decreased so as to prevent the accuracy of the irradiation dose from lowering.

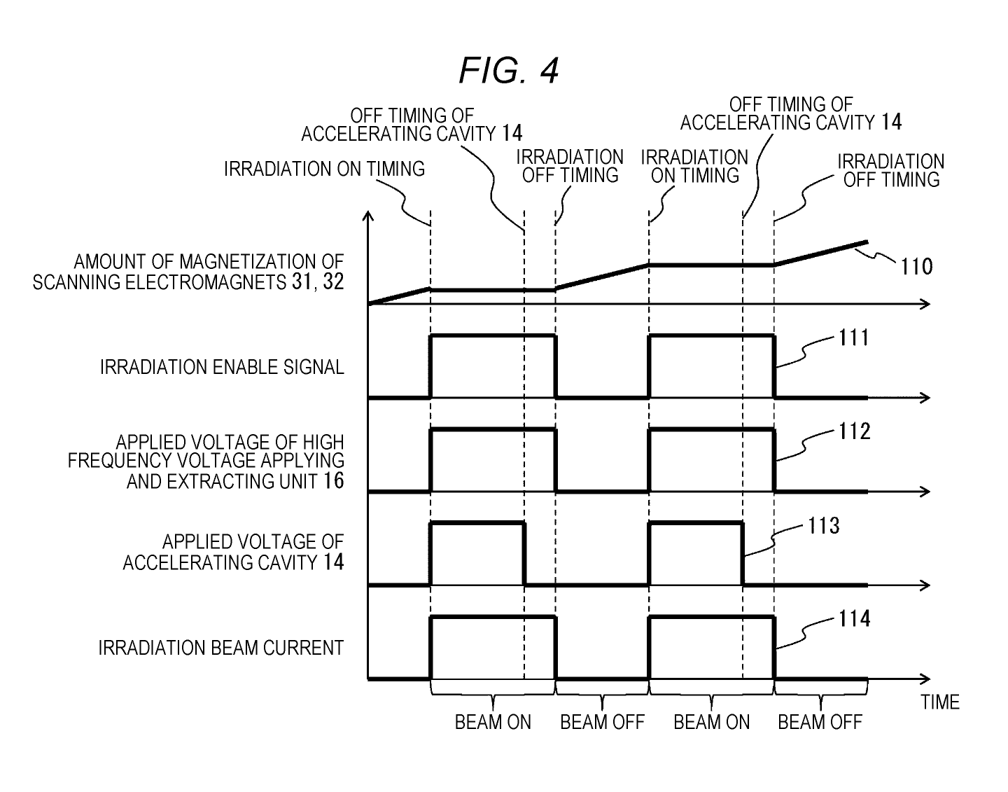

Next, with reference to FIG. 4, a method for controlling the particle beam radiotherapy system according to the present embodiment that improves the accuracy of the irradiation dose in comparison with the scanning irradiation method in the past will be described.

When the accelerating cavity 14 of the synchrotron 10 is turned off, particles of the high-frequency-captured circling beam gradually widen in the longitudinal direction and become a beam that spreads uniformly in the longitudinal direction for the time equal to the period of the synchrotron oscillations while the accelerating cavity 14 is turned on. Although the period of the synchrotron oscillations depends on the layout and operation conditions of components of the synchrotron, the period of the synchrotron oscillations of a proton beam radiotherapy synchrotron is in the range of for example 100 μs to 2000 μs. After the accelerating cavity 14 is turned off until the circling beam spreads uniformly in the longitudinal direction, particles of the circling beam have a density gradient in the longitudinal direction, the circling beam is affected by a force in the longitudinal direction due to the space charge effect. As a result, the momentum of the circling beam slightly changes. Thus, as described above, according to the scanning irradiation method in the past, as the momentum changes due to the space charge effect, after the irradiation stop timing, the circling beam is likely to be extracted.

FIG. 4 is a time chart showing a beam irradiation control method according to the present embodiment implemented by the scanning irradiation method.

In FIG. 4, the horizontal axis represents time and the vertical axis successively represents time changes of the amount of magnetization of the scanning electromagnets 31 and 32 of the particle beam radiotherapy system according to the present embodiment (polygonal line 110), the irradiation enable signal (polygonal line 111), the applied voltage of the high frequency voltage applying and extracting unit 16 (polygonal line 112), the applied voltage of the accelerating cavity 14 (polygonal line 113), and the irradiation beam current (polygonal line 114).

As indicated by the polygonal line 113 shown in FIG. 4, according to the present embodiment, like the scanning irradiation method shown in FIG. 3, the applied voltage of the accelerating cavity 14 is turned off in synchronization with the off state of the beam extraction. However, the beam acceleration high frequency control section 50a of the control unit 50 turns off the accelerating cavity 14 before the irradiation enable signal is turned off.

To implement this control, the control unit 50 successively compares the irradiation dose for the current irradiation spot measured by the dose monitor 33 with the target dose for the current irradiation spot. When the irradiation dose exceeds the target dose by a predetermined ratio, the beam acceleration high frequency control section 50a turns off the accelerating cavity 14. The ratio of the irradiation dose to the target dose with which the accelerating cavity 14 is turned off is set by the technician or the user of the particle beam radiotherapy system so that the elapsed time after the accelerating cavity 14 is turned off (off timing of the accelerating cavity 14) until the irradiation dose reaches the target dose (irradiation off timing) nearly becomes the period of the synchrotron oscillations of particles of the circling beam.

The foregoing control allows the high frequency voltage of the high frequency voltage applying and extracting unit 16 to be continuously applied to the circling beam while the irradiation enable signal is turned on even after the accelerating cavity 14 is turned off. Thus, the beam current extracted from the synchrotron 10 does not change when the accelerating cavity 14 is turned off. On the other hand, since the time equal to the period of the synchrotron oscillations has elapsed after the off timing of the accelerating cavity 14 until the irradiation off timing, the distribution of particles of the circling beam in the longitudinal direction becomes uniform in the irradiation off timing. Thus, according to the present embodiment, since the momentum of particles of the circling beam does not change due to the space charge effect after the irradiation off timing, the amount of the circling beam extracted from the synchrotron 10 decreases after the irradiation off timing. As a result, the accuracy of the irradiation dose can be improved in comparison with the method in the past.

In the particle beam radiotherapy system according to the present embodiment, the off timing of the accelerating cavity 14 is set so that the elapsed time after the off timing of the accelerating cavity 14 until the irradiation off timing becomes equal to the period of the synchrotron oscillations. Alternatively, the elapsed time after the accelerating cavity 14 is turned off until the irradiation off timing may be shorter or longer than the period of the synchrotron oscillations.

For example, when the elapsed time after the off timing of the accelerating cavity 14 until the irradiation off timing is equal to or greater than ¼ of the period of the synchrotron oscillations of particles of the circling beam, the effect of the present embodiment in which the accuracy of the irradiation dose is improved can be derived. After the off timing of the accelerating cavity 14, when the time equal to ¼ of the period of the synchrotron oscillations elapses, particles having high momentum that distribute at a phase near the center of a high frequency bucket in the longitudinal direction and particles having low momentum move to an end of the high frequency bucket. Thus, since the density gradient in the longitudinal direction of particles of the circling beam decreases, the influence of the space charge effect weakens. After the irradiation off timing, the amount of the circling beam extracted from the synchrotron 10 decreases. As a result, the accuracy of the irradiation dose can be improved in comparison with the method in the past.

In the particle beam radiotherapy system according to the present embodiment, the irradiation dose for the current irradiation spot is compared with the target dose for the current irradiation spot. When the irradiation dose exceeds the target dose of irradiation by a predetermined ratio, the accelerating cavity 14 is turned off. Alternatively, after the irradiation has been started, when a predetermined time has elapsed, the accelerating cavity 14 may be turned off.

In this case, the control unit 50 determines the time after the irradiation is started until the accelerating cavity 14 is turned off depending on the target dose for the current irradiation spot and the amount of the circling beam so that the elapsed time after the off timing of the accelerating cavity 14 until the irradiation off timing becomes nearly equal to the period of the synchrotron oscillations.

In addition, in the particle beam radiotherapy system according to the present embodiment, the high frequency voltage applying unit 16 is a beam extraction unit that extracts the beam from the synchrotron 10. Alternatively, the beam extraction unit that extracts a beam from the synchrotron 10 may be a quadrupole magnet. When the quadrupole magnet is used as the beam extraction unit, the quadrupole magnet 12 that adjusts the tune of the circling beam may be used in common with the beam extraction unit. Alternatively, a quadrupole magnet for beam extraction may be disposed in the synchrotron 10. When the quadrupole magnet for beam extraction is disposed in the synchrotron 10, an air-core quadrupole magnet may be used so as to rapidly change the amount of magnetization.

When a quadrupole magnet is used as a beam extraction unit, while the irradiation enable signal is turned on, the amount of magnetization of the quadrupole magnet that extracts the circling beam is changed so that the horizontal tune of the circling beam approaches the resonance line with which the circling beam is extracted. While the irradiation enable signal is turned off, the amount of magnetization of the quadrupole magnet that extracts the circling beam is not changed or the amount of magnetization of the quadrupole magnet that extracts the circling beam is changed so that the horizontal tune of the circling beam goes away from the resonance line with which the circling beam is extracted.

In the particle beam radiotherapy system according to the present embodiment, the beam extraction unit that extracts the circling beam from the synchrotron 10 may be an energy amount changing unit that changes the momentum of particles of the circling beam. If the horizontal chromaticity of the synchrotron 10 is not 0, when the momentum of the circling beam changes, the horizontal tune of the circling beam changes. Thus, when the horizontal chromaticity of the synchrotron 10 is set to an appropriate value, only if the momentum of the circling beam increases, the horizontal tune of the circling beam is approached to the resonance line so as to extract the circling beam from the synchrotron 10.

When the energy changing unit is used as the beam extraction unit, while the irradiation enable signal is turned on, the momentum of the circling beam is changed so that the horizontal tune of the circling beam approaches the resonance line with which the circling beam is extracted. While the irradiation enable signal is turned off, the momentum of the circling beam is not changed or the momentum of the circling beam is changed so that the horizontal tune of the circling beam goes away from the resonance line with which the circling beam is extracted. The energy changing unit is for example a betatron core that accelerates or decelerates the circling beam with the induced electromotive force.

In addition, the beam acceleration high frequency control section 50a of the control unit 50 in the particle beam radiotherapy system according to the present embodiment gradually decreases the high frequency voltage applied to the accelerating cavity 14 instead of turning off the amplitude value of the high frequency voltage applied to the accelerating cavity 14 while the high frequency voltage applying unit 16 performs the extraction control for the charged particle beam.

### Second Embodiment

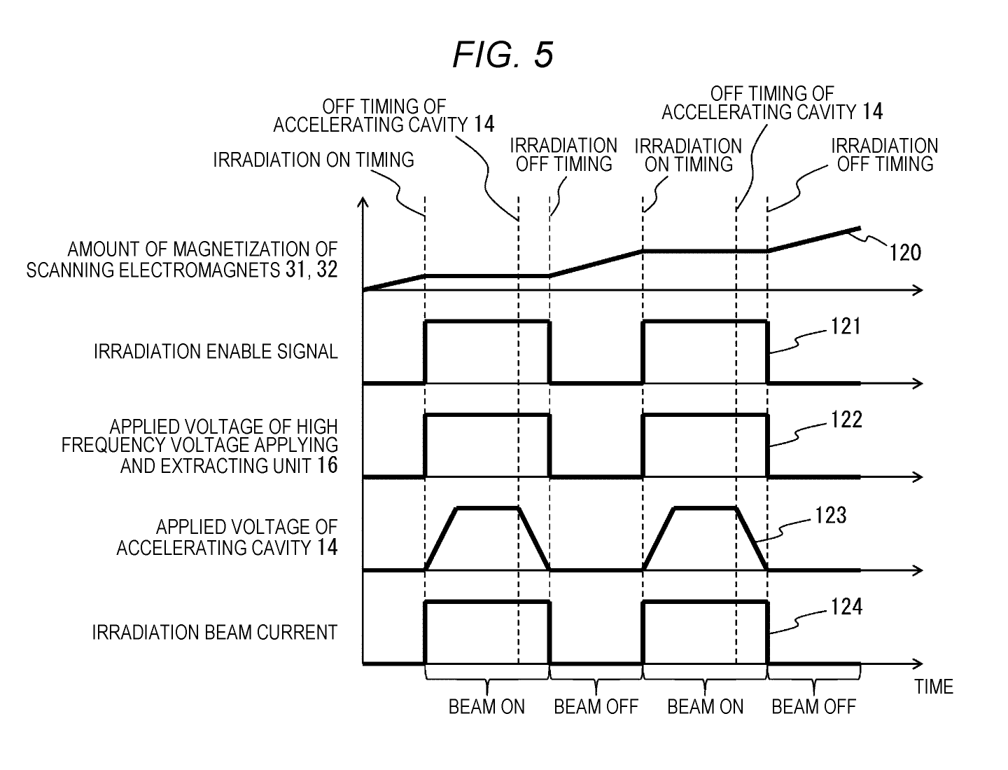

Next, with reference to FIG. 5, a particle beam radiotherapy system and a control method for the particle beam radiotherapy system according to a second embodiment of the present invention will be described. According to the present embodiment, a particle beam radiotherapy system that improves the accuracy of the irradiation dose and that reduces the costs of components of the system will be described.

The particle beam radiotherapy system according to the present embodiment is approximately the same as the particle beam radiotherapy system according to the first embodiment except for how the beam acceleration high frequency control section 50a of the control unit 50 increases or decreases the high frequency voltage applied to the accelerating cavity 14 to the circling beam. Since the other structure of the particle beam radiotherapy system according to the present embodiment is approximately the same as that according to the first embodiment, their description will be omitted.

In the particle beam radiotherapy system according to the first embodiment, the beam acceleration high frequency control section 50a of the control unit 50 rapidly increases the amplitude value of the applied voltage of the accelerating cavity 14 when the accelerating cavity 14 is turned on in synchronization with the beam irradiation on state. In addition, the beam acceleration high frequency control section 50a rapidly decreases the amplitude value of the applied voltage of the accelerating cavity 14 before the beam irradiation off state.

When the amplitude value of the applied voltage of the accelerating cavity 14 to the circling beam that circles in the synchrotron 10 is rapidly and repeatedly increased or decreased, the circling beam is disturbed in the longitudinal direction and thereby the momentum of the circling beam widens (hereinafter referred to as the dispersion of momentum). When the dispersion of momentum of the circling beam increases, the dispersion of momentum of the extracted beam of the synchrotron 10 also increases.

Beam sizes sx (horizontal direction) and sy (vertical direction) in the high energy beam transport 20 are represented by Formulas 1 and 2 where εx and εy are emittances of an extracted beam, Δp/p is dispersion of momentum, βx and βy are Twiss parameters where the beam size is calculated in the high energy beam transport 20, and ηx and ηy are dispersions.

$$\begin{matrix}[ Mathematical Formula 1 ] & \\ {s}_{x} = \sqrt{{ɛ}_{x} {β}_{x} + {( {η}_{x} \frac{Δ p}{p} )}^{2}} & ( 1 ) \\ [ Mathematical Formula 2 ] & \\ {s}_{y} = \sqrt{{ɛ}_{y} {β}_{y} + {( {η}_{y} \frac{Δ p}{p} )}^{2}} & ( 2 )\end{matrix}$$

As the emittances εx and εy of the extracted beam, values containing for example 90% of particles of the circling beam are used. Likewise, as the dispersion of momentum Δp/p, one side of the range containing for example 90% of particles of the circling beam is used. A suffix x represents the horizontal direction, whereas a suffix y represents the vertical direction.

Formulas 1 and 2 reveal that when the dispersion of momentum of the extracted beam increases, the beam size in the high energy beam transport 20 may increase. Thus, in the particle beam radiotherapy system according to the first embodiment, when the accelerating cavity 14 is turned on and off in synchronization with the beam irradiation on/off states, it is concerned that the beam size in the high energy beam transport 20 increases.

The beam passes through a vacuum duct (not shown) of the high energy beam transport 20. The inner diameter of the vacuum duct needs to be greater than the beam size in the high energy beam transport 20 so that particles of the beam that pass through the vacuum duct do not collide with an inner wall of the vacuum duct and are not lost.

When the accelerating cavity 14 is turned on and off, if the beam size in the high energy beam transport 20 increases, the vacuum duct needs to have an inner diameter greater than the increased beam size. Thus, the manufacturing cost of the vacuum duct increases in comparison with the control section in which the beam size does not increase. In addition, when the beam size in the high energy beam transport 20 increases, a region of the beam to which the magnetic field is applied increases in the high energy beam transport 20. Thus, it is concerned that the magnetic poles of the electromagnets that compose the high energy beam transport 20 need to be enlarged and thereby the manufacturing cost of the electromagnets increases.

Thus, to prevent the beam size in the high energy beam transport 20 from increasing in synchronization with the on/off states of the accelerating cavity 14, the beam acceleration high frequency control section 50a of the control unit 50 in the particle beam radiotherapy system according to the present embodiment performs control to gradually decrease the amplitude value of the applied voltage of the accelerating cavity 14 while the high frequency voltage applying unit 16 performs the extraction control for the charged particle beam, and the beam acceleration high frequency control section 50a performs control to gradually increase the amplitude value after the high frequency voltage applying unit 16 starts the extraction control as indicated by the polygonal line 123 shown in FIG. 5.

FIG. 5 is a time chart showing a beam irradiation control method according to the present embodiment implemented by the scanning irradiation method. In FIG. 5, the horizontal axis represents time and the vertical axis successively represents time changes of the amount of magnetization of the scanning electromagnets 31 and 32 of the particle beam radiotherapy system according to the present embodiment (polygonal line 120), the irradiation enable signal (polygonal line 121), the applied voltage of the high frequency voltage applying and extracting unit 16 (polygonal line 122), the applied voltage of the accelerating cavity 14 (polygonal line 123), and the irradiation beam current (polygonal line 124).

More specifically, like the first embodiment, in synchronization with the beam irradiation on and off states, the control unit 50 according to the present embodiment repeatedly turns on and off the accelerating cavity 14.

Since the period of the synchrotron oscillations is reversely proportional to the square root of the amplitude value of the applied voltage of the accelerating cavity 14, while the amplitude value of the applied voltage of the accelerating cavity 14 increases, the period of the synchrotron oscillations decreases. While the amplitude value of the applied voltage of the accelerating cavity 14 decreases, the period of the synchrotron oscillations increases. According to the present embodiment, the period of the synchrotron oscillations represents the time after the amplitude value of the applied voltage of the accelerating cavity 14 has increased until the amplitude value of the applied voltage of the accelerating cavity 14 is decreasing, namely while the amplitude value of the applied voltage of the accelerating cavity 14 is maximum.

According to the present embodiment, when the accelerating cavity 14 is turned off, the beam acceleration high frequency control section 50a of the control unit 50 gradually decreases the amplitude value of the applied voltage of the accelerating cavity 14 before the irradiation off timing so that the time for which the applied voltage of the accelerating cavity 14 decreases becomes nearly equal to the period of the synchrotron oscillations of particles of the circling beam. In addition, when the beam irradiation is started and the accelerating cavity 14 is turned on, the beam acceleration high frequency control section 50a of the control unit 50 gradually increases the applied voltage of the accelerating cavity 14 so that the time for which the applied voltage of the accelerating cavity 14 increases becomes nearly equal to the period of the synchrotron oscillations.

Like the first embodiment, the control unit 50 according to the present embodiment successively compares the irradiation dose for the current irradiation spot measured by the dose monitor 33 with the target dose for the current irradiation spot. When the irradiation dose matches the target dose by a predetermined ratio, the beam acceleration high frequency control section 50a controls the synchrotron 10 to start decreasing the amplitude value of the applied voltage of the accelerating cavity 14.

The ratio of the irradiation dose to the target dose with which the amplitude value of the applied voltage of the accelerating cavity 14 is decreased is set appropriately by the technician or the user of the particle beam radiotherapy system so that the elapsed time after the amplitude value is decreasing until the irradiation is turned off becomes nearly equal to the period of the synchrotron oscillations of particles of the circling beam.

When the control unit 50 according to the present embodiment turns on the accelerating cavity 14, the beam acceleration high frequency control section 50a controls the accelerating cavity 14 so that the amplitude value of the applied voltage of the accelerating cavity 14 increases for the time equal to the period of the synchrotron oscillations.

Like the first embodiment, the display unit 51 is connected to the control unit 50. The display unit 51 displays time changes of the beam irradiation enable signal and time changes of the applied voltage of the accelerating cavity 14 on the same screen. With reference to these signals displayed on the display unit 51, the technician or the user of the particle beam radiotherapy system can check that the amplitude value of the applied voltage of the accelerating cavity 14 is decreasing before the irradiation off timing and that the time for which the applied voltage of the accelerating cavity 14 increases or decreases is nearly equal to the period of the synchrotron oscillations.

Like the first embodiment, the foregoing control allows the high frequency voltage of the high frequency voltage applying unit 16 to be continuously applied to the circling beam while the irradiation enable signal is turned on. Thus, when the accelerating cavity 14 is turned off, the beam current extracted from the synchrotron does not change. In addition, since the time for which the amplitude value of the applied voltage of the accelerating cavity 14 decreases is nearly equal to the period of the synchrotron oscillations, after the applied voltage of the accelerating cavity 14 becomes 0, particles of the circling beam distribute equally in the longitudinal direction. Thus, in the particle beam radiotherapy system according to the present embodiment, while the irradiation is turned off, particles of the circling beam distribute equally in the longitudinal direction. After the irradiation off timing, the space charge effect does not cause the momentum of particles of the circling beam to change. Moreover, according to the present embodiment, while the irradiation is turned off, since the applied voltage of the accelerating cavity 14 is nearly 0, after the irradiation off timing, the synchrotron oscillations do not cause the momentum of particles of the circling beam to change.

Thus, in the particle beam radiotherapy system according to the present embodiment, after the irradiation off timing, since the momentum of particles of the circling beam does not change, like the first embodiment, after the irradiation off timing, the amount of the circling beam extracted from the synchrotron 10 decreases. As a result, the accuracy of the irradiation dose can be improved.

In addition, according to the present embodiment, when the control unit 50 turns on the accelerating cavity 14, the control unit 50 controls the accelerating cavity 14 so that the amplitude value of the applied voltage of the accelerating cavity 14 increases for the time nearly equal to the period of the synchrotron oscillations. When the control unit 50 turns off the accelerating cavity 14, the control unit 50 controls the accelerating cavity 14 so that the amplitude value of the applied voltage of the accelerating cavity 14 decreases for the time nearly equal to the period of the synchrotron oscillations.

Thus, when the momentum and phase of particles of the circling beam in the longitudinal direction change in synchronization with the on/off states of the accelerating cavity 14, particles of the circling beam adiabatically change. As a result, although the accelerating cavity 14 is repeatedly turned on and off, the momentum of the circling beam is prevented from largely dispersing. Consequently, according to the present embodiment, since the momentum of the extracted beam of the synchrotron 10 is prevented from largely dispersing, the size of the beam that passes through the high energy beam transport 20 is prevented from increasing. Thus, the manufacturing costs of the vacuum duct and the electromagnets that compose the high energy beam transport 20 can be reduced.

In the particle beam radiotherapy system according to the present embodiment, the control unit 50 controls the applied voltage of the accelerating cavity 14 so that the time for which the applied voltage of the accelerating cavity 14 increases or decreases becomes nearly equal to the period of the synchrotron oscillations of particles of the circling beam. Alternatively, the time for which the applied voltage of the accelerating cavity 14 increases or decreases may be shorter or longer than the period of the synchrotron oscillations.

For example, when the time for which the applied voltage of the accelerating cavity 14 increases or decreases is equal to or greater than ¼ of the period of the synchrotron oscillations of particles of the circling beam, the size of the beam in the high energy beam transport 20 can be prevented from increasing as an effect of the present embodiment. In other words, when the time for which the applied voltage of the accelerating cavity 14 increases or decreases is ¼ of the period of the synchrotron oscillations, the longitudinal speed of particles of the high-frequency-captured circling beam at the boundary of the high frequency bucket becomes nearly equal to the longitudinal speed in the phase space in longitudinal direction.

As indicated by the polygonal line 123 shown in FIG. 5, the amplitude value of the applied voltage of the accelerating cavity 14 is linearly increased and decreased. The amplitude value of the applied voltage of the accelerating cavity 14 may not be linearly increased and decreased. Alternatively, the amplitude value of the applied voltage of the accelerating cavity 14 may be increased and decreased exponentially, logarithmically, quadratically, or stepwise.

In addition, according to the present embodiment, like the first embodiment, the beam extraction unit that extracts a beam from the synchrotron 10 may be a quadrupole magnet or an energy changing unit that changes the momentum of particles of the circling beam (for example, betatron core).

### <Others>

The present invention is not limited to the foregoing embodiments. The present invention includes various modifications of the embodiments. For example, the foregoing embodiments are described so that the present invention can be easily understood. The present invention is not limited to embodiments that include all components that have been described. Part of the structure of one embodiment may be substituted with part of the structure of another embodiment. In addition, the structure of one embodiment may be added to the structure of another embodiment. Moreover, another structure may be added, deleted, and/or substituted to, from, with the other structure. Furthermore, control lines and information lines regarded to be necessary are illustrated. Thus, all control lines and information lines of the product may not be indicated. Actually, most of the structural elements are mutually connected.

According to the foregoing embodiments, the irradiation field is formed by the spot scanning irradiation method. Alternatively, the irradiation field may be formed by the luster scanning method or the scatterer irradiation method.

What is claimed is:

1. A particle beam radiotherapy system, comprising:
a synchrotron that accelerates and emits a charged particle beam;
an irradiation unit that irradiates an irradiation target with the charged particle beam emitted from the synchrotron;
a beam transport that transports the charged particle beam from the synchrotron to the irradiation unit; and
a control unit that controls the synchrotron, the beam transport, and the irradiation unit,
wherein the synchrotron includes an accelerating cavity that accelerates the charged particle beam at a high frequency acceleration voltage until the charged particle beam has predetermined energy and an extraction unit that extracts the charged particle beam from the synchrotron, and
wherein the control unit includes a beam acceleration high frequency control section that decreases an amplitude value of a high frequency voltage applied to the accelerating cavity while the extraction unit performs extraction control for the charged particle beam.

2. The particle beam radiotherapy system according to claim 1,
wherein a distribution of an irradiation dose for the irradiation target is formed by a scanning irradiation method.

3. The particle beam radiotherapy system according to claim 1,
wherein the beam acceleration high frequency control section of the control unit gradually decreases the amplitude value of the high frequency voltage applied to the accelerating cavity while the extraction unit performs extraction control for the charged particle beam and the control unit gradually increases the amplitude value of the high frequency voltage applied to the accelerating cavity after the extraction unit starts performing the extraction control for the charged particle beam.

4. The particle beam radiotherapy system according to claim 3,
wherein each of time after the beam acceleration high frequency control section of the control unit starts decreasing the amplitude value of the high frequency voltage applied to the accelerating cavity as a decrease start time until stopping decreasing the amplitude value as a decrease stop time and time after the beam acceleration high frequency control section starts increasing the amplitude value of the high frequency voltage applied to the accelerating cavity as an increase start time until stopping increasing the amplitude value as an increase stop time is longer than ¼ of a period of synchrotron oscillations of particles of the charged beam.

5. The particle beam radiotherapy system according to claim 4,
wherein each of the time after the decrease start time until the decrease stop time and the time after the increase start time until the increase stop time is equal to the period of the synchrotron oscillations of the particles of the charged beam.

6. The particle beam radiotherapy system according to claim 1,
wherein the extraction unit is a high frequency voltage applying unit that applies a high frequency voltage in a direction perpendicular to a longitudinal direction of the charged particle beam to the charged particle beam that circles in the synchrotron.

7. The particle beam radiotherapy system according to claim 1,
wherein the extraction unit is a quadrupole magnet that converges or diverges the charged particle beam that circles in the synchrotron on a plane perpendicular to the longitudinal direction of the charged particle beam.

8. The particle beam radiotherapy system according to claim 1,
wherein the extraction unit is an energy changing unit that changes kinetic energy of the charged particle beam that circles in the synchrotron.

9. A method for controlling a particle beam radiotherapy system including
a synchrotron that accelerates and emits a charged particle beam;
an irradiation unit that irradiates an irradiation target with the charged particle beam emitted from the synchrotron;
a beam transport that transports the charged particle beam from the synchrotron to the irradiation unit; and
a control unit that controls the synchrotron, the beam transport, and the irradiation unit,
the method comprising:
causing an amplitude value of a high frequency voltage applied to an accelerating cavity of the synchrotron to decrease while the extraction unit is performing extraction control for the charged particle beam from the synchrotron when the synchrotron emits the beam.
